(19) United States
(12) Patent Application Publication
(10) Pub. No.: US 2021/0321696 A1
(43) Pub. Date: Oct. 21, 2021

(54) COVID MASK

(71) Applicant: First Step Holdings, LLC, Tucson, AZ (US)

(72) Inventors: Jeffry B. SKIBA, Chandler, AZ (US); Scott N. SHEFTEL, Tucson, AZ (US)

(21) Appl. No.: 16/941,371

(22) Filed: Jul. 28, 2020

Related U.S. Application Data
(60) Provisional application No. 63/012,262, filed on Apr. 20, 2020.

(51) Int. Cl. A41D 13/11; A41D 31/30; D06M 11/83; B01D 53/32; B01D 39/08; A61L 9/16; A62B 23/02

(52) U.S. Cl. CPC A41D 13/1192; A61L 2209/14; D06M 11/83; B01D 53/326; B01D 53/323; B01D 39/086; A61L 9/16; A62B 23/025; B01D 2239/0241; B01D 2239/0407; B01D 2239/0442; B01D 2239/0609; B01D 2239/0613; B01D 2239/0645; A41D 31/30

(57) ABSTRACT

Disclosed is a method and device for inactivating or killing airborne bacteria, fungi, molds and viruses by passing the air through a filter material formed of a fabric having particles of zinc disposed in discrete physically isolated locations, wherein the particles of zinc and oxygen from the air form half cells of a battery whereby to create an electrical field that inactivates or kills airborne bacteria, fungi, molds and viruses passing through the filter material.

### CROSS REFERENCE TO RELATED PATENT APPLICATION

This application claims benefit to U.S. Provisional Patent Application Ser. No. 63/012,262, filed Apr. 20, 2020, the contents of which are incorporated herein by reference.

### BACKGROUND OF THE DISCLOSURE

The present disclosure relates to filter media or materials, and in particular to filter materials for exchanging or filtering air. The disclosure has particular utility in connection with filter materials for forming facial coverings, i.e., face masks, and will be described in connection with such utility, although other utilities are contemplated including, for example for forming air handling filters for people movers, i.e., automobiles, trucks, buses, trains, ships and planes, as well as for forming filters for air handling equipment for buildings. Facial coverings, i.e., facemasks or facial mask inserts or replaceable cartridges or PPE masks, fabrics, or barriers including gloves, are considered by health experts to be a first line of defense against transmission of various diseases. The use of face masks has become a critical tool in fighting spread of disease during the current Covid-19 pandemic. However, for many people, the use of face masks may result in skin irritation or allergies. This can be a significant problem, particularly for healthcare and essential workers who must wear face masks all day.

One reason irritation occurs is that the face masks do not allow free airflow to the face, since face masks are designed to be worn closely fitting to the face. Thus, when the wearer breathes, moisture or oils may accumulate and become trapped on the face. The resulting dark, warm environment can cause skin issues such as acne or “mask rash”. In addition, face masks and other facial coverings can irritate the skin simply by rubbing against it, or by exposing the skin to allergens. Also, the type of material and its contact with the skin may have a negative effect resulting secondary irritation or contact dermatitis.

In our earlier U.S. Pat. Nos. 9,192,761 and 9,707,172, we describe devices for treating hyperhidrosis. Hyperhidrosis is a medical condition in which patients experience excessive sweating. Excessive sweating can lead to a patient's physical and societal discomfort, and may also lead to skin irritation and other skin problems. More particularly, the device described in our aforesaid prior US patents, comprises a fabric having zinc particles disposed on at least a portion of the fabric, wherein the fabric is configured to contact a body surface such that the zinc particles come in contact with a skin surface, whereby to create a zinc-oxide battery which effectively treats hyperhidrosis. Also, as reported in our US Publication No. US 2019/016190, our prior patented devices also may be used to treat a variety of other conditions suffered by both humans and animals, including for example, neuropathic pain (including peripheral artery disease and neuropathy, surgical rehabilitation including joint surgery rehabilitation, surgery convalescence including joint surgery rehabilitation and soft tissue healing; physical therapy including muscle and tendon healing and stroke rehabilitation. Our aforesaid patented devices also are said to enhance athletic performance, endurance and to promote faster recovery after exertion along with less muscle discomfort and fatigue.

As described in our aforesaid patents and pending applications the particles of zinc, zinc oxides or zinc salts are carried on a surface of a fabric as a plurality of dots or lines in a specific pattern that positions the zinc reservoirs in discrete locations, each location separated by a distance. When this zinc-carrying fabric is placed with the zinc particles in contact with the skin of a person or animal, the zinc particles separation and configuration couples with oxygen and moisture at the skin surface to create a zinc-oxygen battery which produces an electric current at the skin.

The mechanism is as follows: The zinc-carrying fabric pattern acts as a half-cell anode and the oxygen partially supplied by circulation at the skin surface acts as a half-cell cathode physically separated to allow electric fields to exist. The human or animal's body contributes moisture, which completes the circuit to allow current to flow as a function of impedance within the tissues. The completed circuit creates a redox reaction with oxidation of the zinc and reduction of the oxygen (2Zn+O₂→2ZnO). The oxygen is ambient or replenished with the circulating blood oxygen (partial pressure of oxygen diffusing through the skin) at the skin's surface. As long as there is zinc and oxygen, a field exists and as long as there is a conductive tissue, current will flow.

Microcurrent stimulation is a known phenomena in the range of millionths of an ampere. Humans and other animals have inherent electrical (microcurrent) properties that drive and maintain their bodies. Cells communicate with one another via complex neuro pathways generated and maintained by biochemical reactions that create electrical activity and these endogenous point charges, electric fields and electric currents all have a function in cell signaling such as migration patterns, expression of reactive oxygen species, and regulation of gene expressions. The body generates electrical fields in vital organs such as the heart and brain that are easily measured with instruments such as EEG (electroencephalogram), and EKG (electrocardiogram). The electrophysiology of the human body indicates that cells and organs possess an electrical nature. Studies of electric field and microcurrent stimulation have been well documented for decades. The effect on the human body is evident both clinically and on a cellular level. Physiologic studies document increased capillary density, enhanced blood flow and tissue oxygenation, as well as an enhanced cellular response with increased protein synthesis, amino acid transport and increased ATP (mitochondrial energy) synthesis. In addition to amplifying critical cellular functions within the cell, microcurrent also may increase local cellular absorption of nutrients and facilitates waste elimination, a critical component of muscle performance and recovery.

While some doses of electricity stimulate cellular activity, specific doses can suppress or inhibit cellular function. An example of inhibitory activity is seen with the effect of electrical current on sweat production and bacterial growth. The efficacy of applying external electrical current to the skin for control of excessive sweating (hyperhidrosis) is historically well documented. This concept is the basis for hyperhidrosis treatments utilizing external battery devices such as marketed under the name Drionics, available from General Medical Company. In addition to reducing sweat gland activity, electrical current inhibits the activity of bacteria and fungi, the organisms responsible for foot odor and athletes foot.

### SUMMARY OF THE DISCLOSURE

As used herein the term “metal” shall include particles of elemental metal, as well as particles of oxides and salts of said metal.

As used herein the term fabric shall include woven fabrics, as well as non-woven fabrics and other types of sheet material including fiber-reinforced paper and various types of composite barrier materials.

We now have found that zinc loaded fabrics made having spaced particles of elemental zinc, zinc oxide and/or zinc salt and certain other metals, such as copper, silver and magnesium, and oxides and salts thereof advantageously can be used to form filter media which can act as a barrier to a variety of airborne pathogens and viruses, i.e., as filter media in PPE Masks, air cleaners and/or filters HVAC units in hospitals and nursing homes, residential and commercial building applications, as well as transportation applications—anywhere air must be cleaned of airborne pathogens or viruses.

An essential element in the design considerations of an air filter media is to minimize static pressure drop across the media. We have found we can provide zinc loaded fabrics that exhibit minimal static pressure drop without compromising zinc oxygen battery formation or efficiency. This feature is especially useful to reduce heat or humidity buildup inside of a PPE mask. The zinc oxide battery also kills COVID and other pathogens, such as H1N1, SARS, MERS and pneumonia, while keeping skin healthy.

In one aspect of the disclosure there is provided a method of inactivating or killing airborne bacteria, fungi, molds and viruses comprising passing the air through a filter material formed of a fabric having particles of zinc disposed in discrete physically isolated locations, wherein the particles of zinc and exhaled oxygen form half cells of a battery. There is moisture in exhaled air that completes the circuit to create an electrical field that inactivates or kills airborne bacterial, fungi, molds and viruses passing through the filter material.

In one embodiment, the fibers are spatially separated within the fabric to set up an electric field as determined by the weave pattern or the knit pattern.

In another embodiment, the fiber surface area of the fabric can be increased by flocking, felting and/or terrying which would allow designs to slow down passage of airborne particles and expose more of the active fibers to the airborne pathogens.

In another and preferred embodiment, the filter material is formed into a face mask as either a single layer or as part of a composite which could include absorbents, water proofed, or other performance fabrics that could increase overall performance.

In yet another embodiment the fabric is formed of threads or filaments having particles of zinc, and threads or filaments having particles of copper, silver or magnesium, woven or with a neutral insulating fiber or thread as part of the weave, knot or non-woven process. The neutral thread is present to separate the active fibers from each other to form electric fields which would not exist if the fabric was coated completely.

In a further embodiment wherein the filter material is a fabric formed of fibers or filaments having particles of zinc, zinc oxide and/or zinc salt, and fibers or filaments having particles of copper, silver or magnesium, copper, silver or magnesium oxide, or copper, silver or magnesium salt wherein the filaments are woven, knitted or thermally fused, and separated at least in part from one another. These fibers may be used to form a fabric or mesh with batteries of several different cell types creating areas of high and low field strengths, of batteries that would exhaust with time while others continue to provide energy. In this embodiment, a high electric field could be designed to last for a period of time while a second electric field construct could initiate when the first dies away, and so forth. Batteries are defined by field strength and capacity. We can control the field strength with the half cell, and the capacity with the amount of zinc, copper, etc within the cell. In a biologic environment we also can use bioresorbable polymers to slowly degrade exposing more of the cell for reaction. An example would be a conical shaped reservoir where the large area of the cone would provide higher capacity and as the cone gets smaller, the field shape changes and the capacity is reduced until more cone is exposed.

In another embodiment oxygen carried by the air passing through the filter material reacts with the zinc to form a half-cell.

In another embodiment oxygen carried by the exhaled air passing through the filter material reacts with the zinc to form a half-cell.

In still another embodiment supplemental oxygen is provided from an external oxygen source selected from hyperbaric oxygen, hydrogen peroxide, or an oxygen concentrator or other.

The present disclosure also provides a filter capable of inactivating or killing airborne bacteria, fungi, molds and viruses, said filter being formed of a material comprising a filter material formed of a fabric having particles of zinc disposed in discrete physically isolated locations, wherein the particles of zinc and oxygen from the air form half cells of a battery whereby to create an electrical field that inactivates or kills airborne bacterial, fungi, molds and viruses passing through the filter material.

In one embodiment, the fibers are spatially separated within the fabric to set up an electric field as determined by the weave pattern or the knit pattern.

In another embodiment the fiber surface area of the fabric is increased by sanding, flocking, felting and/or terrying.

In yet another and preferred embodiment the filter is in the form of a face mask.

In another embodiment, the fabric is formed of threads or filaments having particles of zinc, and threads or filaments having particles of copper, silver or magnesium, copper, silver or magnesium oxide, or copper, silver or magnesium salt, woven or with a neutral insulating fiber or thread as part of the weave, knot or non-woven process.

In still another embodiment, the filter material is a fabric formed of fibers or filaments having particles of zinc, zinc oxide and/or zinc salt, and fibers or filaments having particles of copper, silver or magnesium, copper, silver or magnesium oxide, or copper, silver or magnesium salt wherein the filaments are woven, knitted or thermally fused, and separated at least in part from one another.

In another embodiment the filter is in the form of an air filter configured for use in a people mover.

In still another embodiment the filter is in the form of an air filter for use in air handling equipment for buildings.

An additional feature of our zinc oxygen battery technology is that in contact with the skin, it is highly beneficial and increases the Collagen 1 Collagen 3 ratio, and aids in skin tissue health. Other technologies used to minimize mask rash such as bleach loaded fabrics or copper loaded fabrics do not possess this feature.

### BRIEF DESCRIPTION OF THE DRAWINGS

Further features and advantages of the instant disclosure will be seen from the following detailed description taken in conjunction with the accompanying drawings, wherein

FIG. 1 is a plan view of a filter face mask made in accordance with a preferred embodiment of our disclosure;

FIGS. 2-8 are two dimensional views of fabrics useful in forming filters in accordance with the instant disclosure; and

FIGS. 9A-9E are three dimensional views of fabrics useful in forming filters in accordance with the instant disclosure.

### DETAILED DESCRIPTION OF THE DISCLOSURE

A zinc oxygen battery produces between 0.1 and 1 Volt providing an electric field by design, keeping positive and negative poles slightly separated and not shorted out. This physical separation is essential and creates the unique nature of our electrically active fabric. The amount of fiber, the concentration of the metal on the surface, the particle size of the metal power, the blend of neutral/active fiber, how the fiber is drawn through thermal spinning, the denier or weight of the fiber and the construction of a thread or yarn all may contribute to the battery efficiency and may affect static pressure drop when the fibers are formed into a fabric and used as a filter. Also, our fibers having particles of zinc, zinc oxide or zinc salt, or particles of copper, silver or magnesium could be blown into a melt or non-woven fabric used for form filter media.

Also, physical characteristics of the fiber, weight, size, weave, and other physical characteristic may significantly affect filter efficiency, filter life and static pressure drop. Balancing these characteristics and preparation is critical to forming a useful filter. Zinc and certain other metals may be used to create cells capable of providing voltages; however, it is the creation of electric fields at a small scale within a filter fabric that generates a field at a scale where microbes, bacteria, viruses, and other pathogens will be affected. The use of weaving or knitting allows the design of a variety of patterns that can be mass produced and eventually converted into filter media with active electrical activity. For example, in addition to zinc, an additional dissimilar metal, preferably, copper, silver or magnesium or an oxide or salt thereof, can be added to the fabric to initiate a galvanic cell between the dissimilar fibers physically separated to create an electric field. Field strengths are a function of the half-cell potential of the metals. For example, Zn is −0.75 eV and Ag is +0.76 eV for a theoretical voltage maximum of 1.5 Volts. In a Zn/Cu cell construct the Cu would be at 1.10 volts.

In one embodiment of our disclosure, the fiber can be used to create a woven pattern wherein there is physical separation of metal infused fibers, threads or yarns. This is done using a weave pattern where three fibers or thread are used. One fiber is a positively charged, one fiber is negatively charged and one fiber is a neutral or insulating fiber or thread. In the weaving process the individual active threads can be physically separated by the insulating thread at a distance determined by the thickness and number of insulating threads between the active threads. Active threads can be single or multiple fibers or threads/yarns of a predetermined thickness that will eventually contribute to the weight and feel of the finished fabric. The weave pattern can be loosely woven or tightly woven depending upon the desired electrical output, while balancing static pressure drop. In an embodiment where the woven fabric is to be used for filtration masks the thickness of the fabric, the space between the fibers or threads, the weight of the fabric all contribute to static pressure or the force required to pass air through the woven fabric. A tight weave increases static pressure and a loose weave reduces it.

A woven pattern can be selected that uses a neutral, insulating thread to physically separate the two dissimilar metal active threads. In an alternative embodiment, the two active threads may come into contact with each other at intersections. However, at those intersections, the active electrical field will be lost.

Other techniques such as sanding, felting, terrying, and flocking may be used to increase the available surface area of the active fibers and therefore the amount of active barrier in the filter media. Through these methods, the surface area per unit is increased by lifting the fibers away from the base fabric.

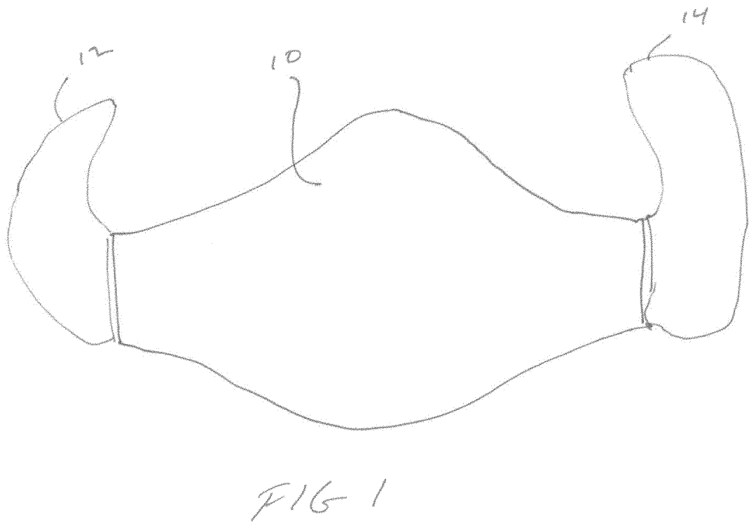

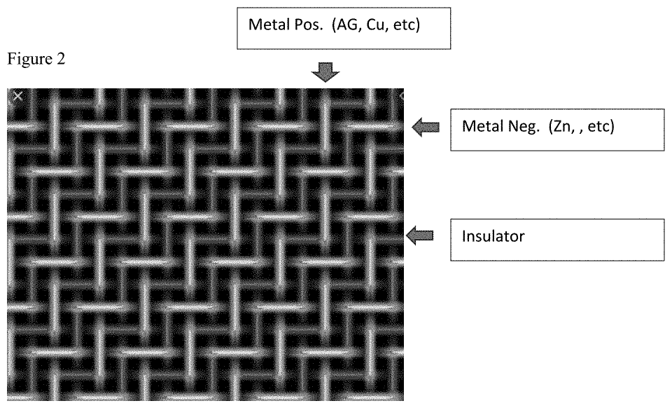

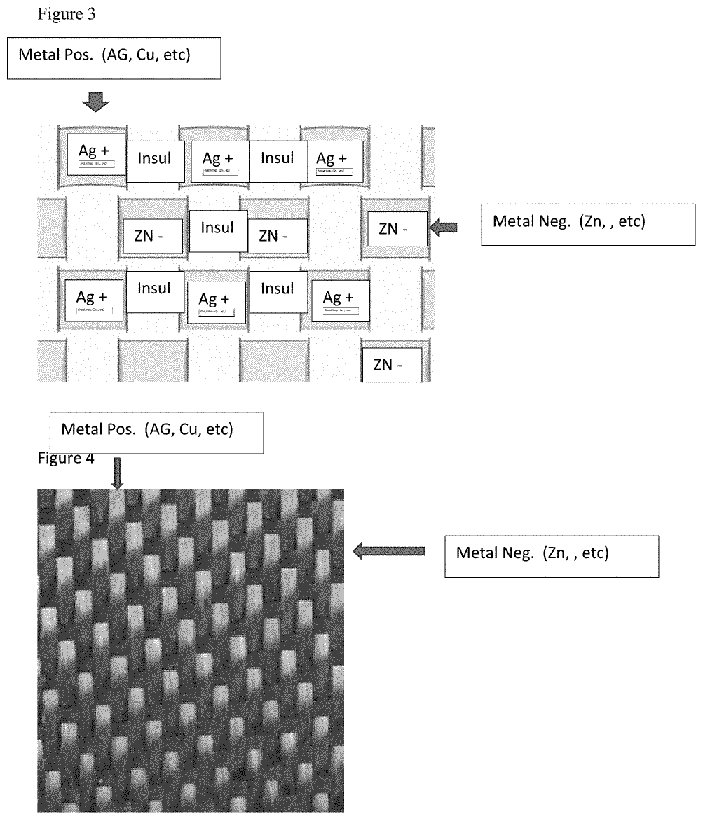

Referring to FIG. 1, a face mask made in accordance with the incident disclosure comprises a main body 10 shaped to fit over the nose and mouth of the wearer. Straps 12, 14 are affixed to the respective distal end of body 10 for fastening the face mask over the ears or behind the head of the wearer. The face mask is similar to take the conventional face mask, except the mask body is formed of a fabric today having elemental zinc particles infused into the fibers of the fabric exposed in part on a surface of the fabric so as to come into contact with the skin of the wearer. The fabric is formed by weaving filaments containing zinc particles spaced from one another, following the teachings of our aforesaid U.S. Pat. Nos. 9,192,761 and 9,707,172, and our published US Application Serial No. 2019/016190 and our PCT Published Application WO 2019/241074, the contents of which are incorporated herein by reference, with filaments optionally containing particles of copper, silver or magnesium spaced from one another, in a basket weave as shown in FIGS. 2 and 3. Preferably the metal particles are zinc particles and have an average particle size of between 1 and 100 nanometers, more preferably 1 to 10 microns, and even more preferably about 5 microns. The metal particles may be printed or bound on the substrate 16, or extruded or melt spun at the time of fiber formation as taught by our aforesaid patents and pending applications. The amount of zinc and the surface area of the zinc or other metal used is a function of particle size and availability to create the battery.

Preferably, but not necessarily, fabric 16 comprises a woven textile, although fabric 16 may be a non-woven textile, a fibrous mesh, a non-fibrous mesh, which may include an adhesive coated textile or fabric, mesh or the like.

As taught in our aforesaid '761 and '172 patents or as described in our pending applications, the metal particles are discontinuously and substantially uniformly distributed on the surface of the fabric, in imaginary spaced lines or lines of dots, across the surface area of the fabric, at least in part. Typically, the lines or lines of dots are evenly spaced at spacings from 0.1 to 3 mm, preferably 0.2 to 2 mm, more preferably 0.3 to 1.5 mm, most preferably 0.5 to 1.0 mm. The concentration of metal such as zinc in the binder or in the extruded fibers that forms the lines or dots determines the amount of metal available for the “battery”. Preferred concentration is 30% of the surface area of the fabric; however, the concentration of zinc may range from about 1% to about 99%. A mixture of binder and zinc metal may be formed as a paste and applied by silk screening e.g., as described in our aforesaid '761 and '172 patents. A 30% by weight zinc-to-binder is preferred for this. The line or dot width and length also determines the amount of metal in the deposition since the wider and longer the line, the more metal is available. Preferred line dots width is 1 mm width but width can vary from 0.1 mm up to 5 mm width. Since the deposition is on a fabric or carried in the adhesive, the amount of binder/metal applied also can be varied. In certain embodiments, the fabric being coated can be coated twice or more times over the same pattern whereupon the thickness of the deposition can be increased as desired. In certain embodiments, the metal deposition area patterns cover from about 10% to about 90% of the surface area of the fabric. In other embodiments, the metal deposition areas cover from about 20% to about 80%, from about 15% to about 75%, from about 25% to about 50%, or from about 30% to about 40% of the surface area of the fabric or anywhere in between. Although FIG. 1 shows the plurality of metal deposition areas 18 substantially uniformly distributed on the surface of the fabric, in other embodiments, the plurality of metal deposition areas may be randomly distributed on the surface of the fabric. Typically, the lines have a thickness of 0.1 to 3 mm, preferably 0.2 to 2 mm, more preferably 0.3 to 1.0, most preferably 0.4 to 0.5 mm. The spaced lines may be continuous and may take various forms including straight, curved and various angular shapes as shown, for example, straight continuous lines; straight broken lines; continuous saw-shaped; continuous wavy lines; broken wavy lines, etc, as described in our aforesaid '761 and '172 patents and our pending applications. The actual shape of the lines is not important. Preferably, but not necessarily, the lines are approximately equal in thickness and are evenly spaced.

In a preferred embodiment the metal particles previously formed by grinding or precipitated out of suspension, and having an average particle size between 1 and 100 nanometers, more preferably 1-10 microns, even more preferably about 5 microns are mixed with a thermal plastic material such as polyethylene in a heated mixing vat 30 to melt the material, and the mixture extruded or melt spun at spinning station 32 to form fibers, having metal particles contained therein. The metals containing fibers may then be cabled or twisted at a cabling station, and woven at a weaving or knitting station into a sheet or cloth. The resulting metal particle impregnated sheet or cloth is cut to size and formed into a mask.

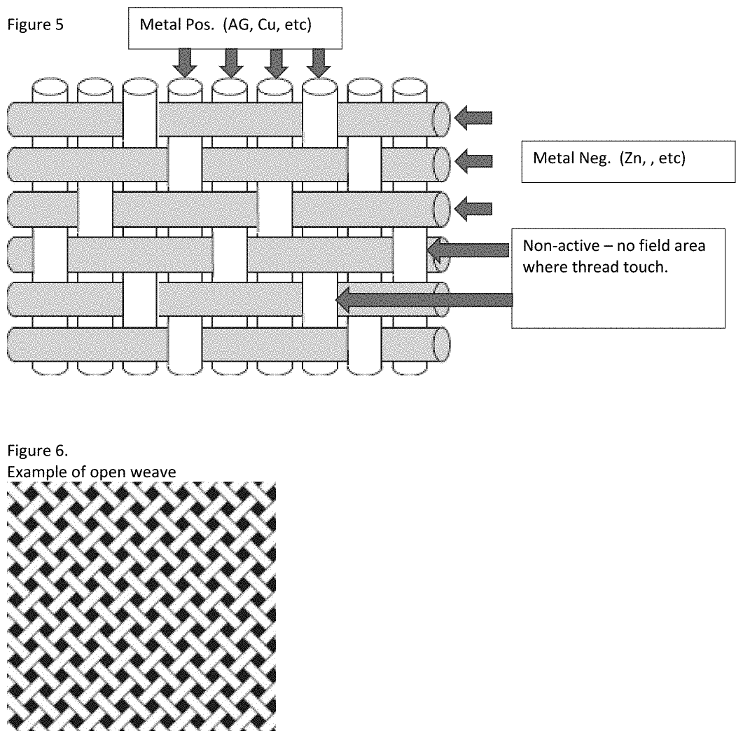

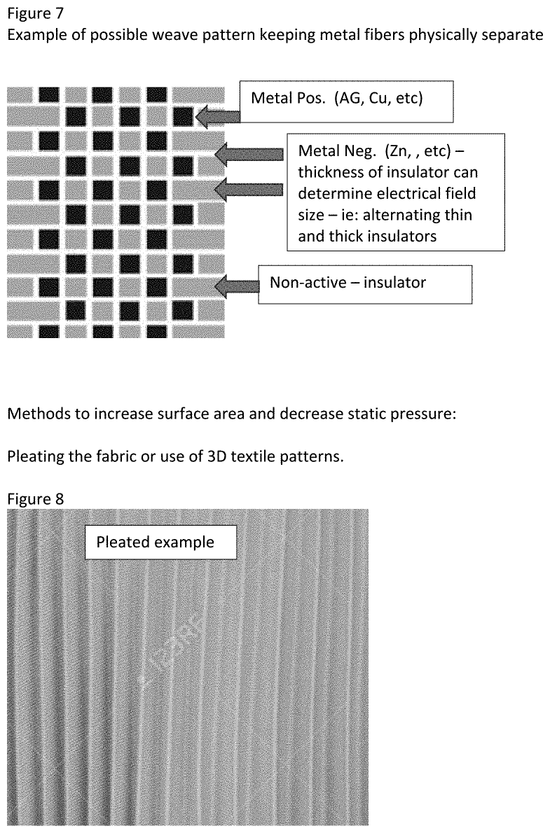

Other weave patterns are possible as illustrated in FIG. 4-8, or 9A-9C. All that is required is that the at least some of the fibers or filaments making up the fabric include particles of elemental zinc, zinc oxide or zinc salt and another elemental metal, metal oxide or metal salt, e.g., of copper, silver or magnesium are separated within the fabric so as to set up an electric field as described in our aforesaid patents and pending application.

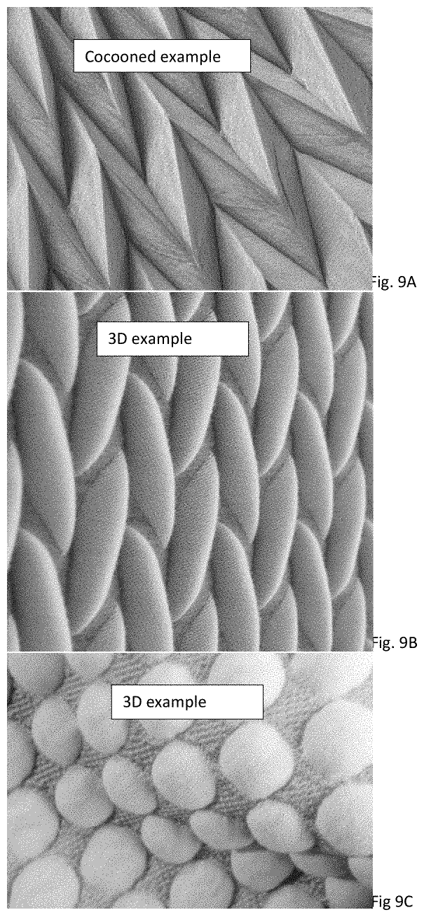

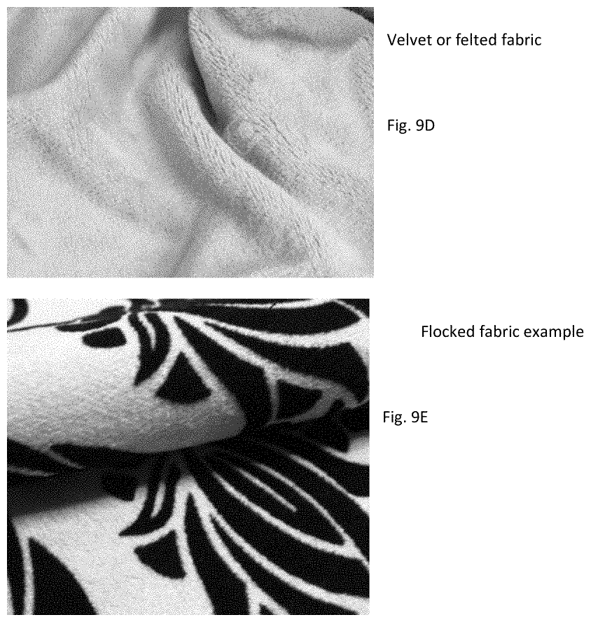

Various changes can be made in the above disclosure without departing from the spirit and scope of our disclosure. For example, the efficiency of the via media as a filter media may be increased by increasing the loading of zinc, zinc oxide or zinc salt or and/or other elemental metal, metal oxide or metal salt; employing finer particles; or by modifying the surface of the fibers of for example, by sanding, felting, flocking or terrying to create an increased surface area/volume, or pleating the fabric so as effectively to increase the surface area of the fiber, by lifting the fiber in part from as the base fabric, as illustrated in FIGS. 9D-9E.

When used as a mask, the filter material of the present disclosure also has several other advantageous effects.

Zinc is a co-factor and is essential to bodily functions. One of its roles is to lessen the formation of damaging free radicals and protects skin's lipids (fats) and fibroblasts the cells that make collagen, one's skin's support structure when skin is exposed to UV light, pollution and other skin-agers. It helps heal and rejuvenate skin. When there is an insult or trauma to the skin, Zinc is essential to the healing process and health of the body. Zinc also is essential to the metabolic process's and health of the body. Zinc lessens the formation of damaging free radicals and protects skin's lipids (fats) and fibroblasts—the cells that make collagen, one's skin's support structure—when skin is exposed to UV light, pollution and other skin-agers. It helps heal and rejuvenate skin. When you cut yourself, zinc goes to work.

Oxygen has a unique effect on the skin because it is important for cellular function and metabolic process. In the presence of Oxygen, the permeability of the skin barrier is enhanced and the skin is more receptive to exogenous stimuli. Also, oxygen has a unique effect on the skin because it opens up our pores, increasing their absorption power. After being exposed to oxygen, the skin starts breathing again and all treatments applied thereafter produce even better results.

Additionally, microcurrents send low-level electrical currents into the wearer's skin that are nearly identical to the body's own natural electrical frequencies, i.e., similar to the effect when physical therapist places electrodes on target areas of the body, or, like getting a microcurrent facial.

Microcurrents also stimulate the wearer's facial muscles for a natural lift, i.e., similar to microcurrent facials which tighten and smooth the muscles and connective tissues in the face by increasing cellular activity, and have been shown to reduce wrinkles, mostly around the forehead area. And, microcurrents also work at the cellular level to literally recharge the wearer's skin back to a more youthful state, and results in increased levels of ATP which speeds cellular metabolism, stimulates protein synthesis, promotes detoxification and reconstitutes collagen and elastin.

Various changes may be made in the foregoing disclosure. For example, two or more dissimilar fabrics may be woven or joined together to create half cell potentials that have a zinc oxygen battery as well as zinc/silver, zinc/copper and/or zinc/magnesium batteries.

While the foregoing disclosure has been primarily directed towards the creation of face mask, the fabric material may be employed for forming other types of filters including, for example, air handling filters for people movers, i.e., automobiles, trucks, buses, trains, ships and planes, as well as for forming filters for air handling equipment.

1: A method of protecting an individual from breathing in live airborne bacteria, fungi, molds and viruses, comprising a providing the individual with a face mask formed of a fabric having particles of zinc disposed in discrete physically isolated locations, wherein the particles of zinc and oxygen and moisture from ambient air and oxygen and moisture from exhaled air and skin of the individual form half cells of a battery which creates an electrical field that inactivates or kills live airborne bacteria, fungi, molds and viruses passing in and out through the face mask as the individual breathes.

2: The method of claim 1, wherein the fabric comprises fibers in a pattern selected from the group consisting of a knit pattern and a weave pattern, wherein the fibers are spatially separated within the fabric to set up an electric field as determined by the weave pattern or the knit pattern.

3: The method of claim 1, wherein the fabric comprises fibers and the fiber surface area of the fabric is increased by a method selected from the group consisting of sanding, flocking, felting and terrying.

4. (canceled)

5: The method of claim 1, wherein the fabric is manufactured in a process selected from the group consisting of weaving, knitting, gluing or non-woven, wherein the fabric comprises threads or filaments having particles of zinc, threads or filaments having particles selected from the group consisting of copper, silver and magnesium, and at least one neutral insulating fiber or at least one neutral insulating thread.

6: The method of claim 1, wherein the fabric is formed of fibers or filaments having particles selected from the group consisting of zinc, zinc oxide and zinc salt, and fibers or filaments having particles selected from the group consisting of copper, silver, magnesium, copper oxide, silver oxide, magnesium oxide, a copper salt, a silver salt, and a magnesium salt, wherein the fabric is manufactured using a process selected from the group consisting of woven, knitted, glued or thermally fused, and wherein at least some of the fibers or filaments of the fabric are separated at least in part from one another.

7. (canceled)

8: The method of claim 1, wherein additional oxygen is provided from an external oxygen source selected from the group consisting of hyperbaric oxygen, hydrogen peroxide, and an oxygen concentrator.

9: A wearable face mask capable of inactivating or killing live airborne bacteria, fungi, molds and viruses, said face mask being formed of a material comprising a filter material formed of a fabric having particles of zinc disposed in discrete physically isolated locations, wherein the particles of zinc and oxygen and moisture from ambient air and oxygen and moisture from exhaled air and skin of a wearer of the filter form half cells of a battery which creates an electrical field that inactivates or kills live airborne bacteria, fungi, molds and viruses passing in or out through the face mask as a wearer of the face mask breathes.

10: The wearable face mask of claim 9, wherein the fabric comprises fibers and a pattern selected from the group consisting of a knit pattern and a weave pattern, wherein the fibers are spatially separated within the fabric to set up an electric field as determined by the weave pattern or the knit pattern.

11: The wearable face mask of claim 9, wherein the fabric comprises fibers and fiber surface area of the fabric is increased by a method selected from the group consisting of sanding, flocking, felting and terrying.

12. (canceled)

13: The wearable face mask of claim 9, wherein the fabric is manufactured in a process selected from the group consisting of weaving, knitting or non-woven, wherein the fabric comprises threads or filaments having particles of zinc, and threads or filaments having particles selected from the group consisting of copper, silver and magnesium, and oxides and salts thereof, with a neutral insulating fiber or at least one neutral insulating thread.

14: The wearable face mask of claim 9, wherein the fabric is formed of fibers or filaments having particles selected from the group consisting of zinc, zinc oxide, zinc salt, and fibers or filaments having particles selected from the group consisting of copper, silver, magnesium, copper oxide, silver oxide, magnesium oxide, a copper salt, a silver salt and a magnesium salt wherein the woven, knitted, glued or thermally fused, and wherein at least some of the fibers or filaments of the fabric separated at least in part from one another.

15. (canceled)

16. (canceled)
